(19) United States
(12) Patent Application Publication
(10) Pub. No.: US 2026/0245723 A1
(43) Pub. Date: Aug. 20, 2026

(54) Digital Display Function to Determine Biometric Information of a User Obtained from an Image of a Display Screen

(71) Applicant: Google LLC, Mountain View, CA (US)

(72) Inventors: Anupam J. Pathak, San Carlos, CA (US); Dongeek Shin, San Jose, CA (US)

(21) Appl. No.: 19/489,566

(22) PCT Filed: Jun. 2, 2023
(86) PCT No.: PCT/US2023/024221

§ 371 Date: Dec. 2, 2025

(51) Int. Cl. G16H 40/67; G06V 10/74

(52) U.S. Cl. CPC G16H 40/67; G06V 10/761; G06V 2201/02; G06V 2201/03

(57) ABSTRACT

A computing device includes one or more memories to store one or more instructions and one or more processors. The one or more processors execute the one or more instructions stored in the one or more memories to: obtain display screen information associated with a display screen of a biometric sensing computing device, obtain an image including the display screen, the image including biometric information associated with a user of the biometric sensing computing device, execute a digital display function based on the display screen information to determine biometric content information from the image corresponding to the biometric information, and store the biometric content information determined by executing the digital display function.

### FIELD

The disclosure relates generally to computing devices. More particularly, the disclosure relates to computing devices which are used to obtain and store biometric information of a user based on an image of a display screen of a biometric sensing computing device.

### BACKGROUND

Some biometric sensing computing devices, such as a pulse oximeter are capable of measuring human biometric information and display the biometric information (e.g., an amount of oxygen saturation) on a display screen (e.g., a light emitting display screen). However, some of the biometric sensing computing devices may delete or stop displaying the biometric measurement when the user removes their finger from the device or after some period of time, and not store the measurements for the user to track over time. Therefore, the user or other users (e.g., medical professionals) may not have access to a history of biometric information measurements.

### SUMMARY

Aspects and advantages of embodiments of the disclosure will be set forth in part in the following description, or can be learned from the description, or can be learned through practice of the example embodiments.

In an example embodiment, a computing device (e.g., a mobile phone, a smartphone, a server computing device, etc.) is provided. The computing device includes one or more memories configured to store one or more instructions and one or more processors configured to execute the one or more instructions stored in the one or more memories to: obtain display screen information associated with a display screen of a biometric sensing computing device, obtain an image including the display screen displaying biometric information associated with a user of the biometric sensing computing device, execute a digital display function based on the display screen information to determine biometric content information from the image corresponding to the biometric information, and store the biometric content information determined by executing the digital display function.

In some implementations, the one or more processors are configured to execute the one or more instructions stored in the one or more memories to: obtain device information associated with the biometric sensing computing device using an image of the biometric sensing computing device, and obtain the display screen information associated with the display screen of the biometric sensing computing device based on the device information.

In some implementations, the one or more processors are configured to execute the one or more instructions stored in the one or more memories to: receive information associated with a user input providing device information associated with the biometric sensing computing device, and obtain the display screen information associated with the display screen of the biometric sensing computing device based on the device information.

In some implementations, the device information includes at least one of a device part number, a device model number, a device name, or a device manufacturer.

In some implementations, the display screen information includes at least one of a resolution of the display screen, a size of the display screen, color information of the display screen, font information of the display screen, a type of the display screen, layout information of the display screen, tolerance information associated with the display screen, or text placement information of the display screen.

In some implementations, the one or more processors are configured to execute the one or more instructions stored in the one or more memories to perform perspective correction with respect to the image displayed on the display screen before executing the digital display function.

In some implementations, the one or more processors are configured to execute the one or more instructions stored in the one or more memories to select one or more digital display functions to determine the biometric content information from the image based on biometric device capabilities associated with the biometric sensing computing device.

In some implementations, the biometric device capabilities associated with the biometric sensing computing device include at least one capability to: generate an electrocardiogram, generate a photoplethysmogram, measure heart rate, measure blood pressure, measure oxygen saturation, measure respiration rate, measure body temperature, measure physical activity, measure a sleep metric, or measure electrical conductance.

In some implementations, the one or more processors are configured to execute the one or more instructions stored in the one or more memories to determine the digital display function to be executed based on whether a display output space provided on the display screen includes a discrete display output space and/or a continuous display output space.

In some implementations, the one or more processors are configured to execute the one or more instructions stored in the one or more memories to, when a first portion of the display output space provided on the display screen includes the discrete display output space, execute a first digital display function with respect to the first portion to: compare the image including the biometric information with each of a plurality of possible discrete values which can be output with respect to the first portion of the display output space, to thereby determine a similarity metric value for each comparison, select a discrete value among the plurality of possible discrete values that is associated with a highest degree of similarity based on the similarity metric values determined from the comparisons, and store the discrete value associated with the highest degree of similarity as the biometric content information corresponding to the biometric information of the user.

In some implementations, the one or more processors are configured to execute the one or more instructions stored in the one or more memories to, when a second portion of the display output space provided on the display screen includes the continuous display output space, execute a second digital display function with respect to the second portion to: determine a type of biometric signal displayed in the image, apply a computer-implemented spline fitting method to reconstruct the biometric signal displayed in the image to obtain a reconstructed biometric signal, based on the type of biometric signal displayed in the image, and store the reconstructed biometric signal as the biometric content information corresponding to the biometric information of the user.

In some implementations, the one or more processors are configured to execute the one or more instructions stored in the one or more memories to, when the reconstructed biometric signal includes one or more missing data portions, apply a computer-implemented spline interpolation method to fully reconstruct the biometric signal, the computer-implemented spline interpolation method being based on the type of biometric signal displayed in the image.

In some implementations, the one or more processors are configured to execute the one or more instructions stored in the one or more memories to, when a first portion of the display output space provided on the display screen includes the discrete display output space, execute a first digital display function to: compare the image including the biometric information with each of a plurality of possible discrete values which can be output with respect to the first portion of the display output space, to thereby determine a similarity metric value for each comparison, identify a discrete value among the plurality of possible discrete values that is associated with a highest degree of similarity based on the similarity metric values determined from the comparisons, when a confidence level associated with the discrete value associated with the highest degree of similarity is less than a threshold confidence level, provide an output requesting feedback from the user confirming a configuration of the display output space, and when the confidence level associated with the discrete value associated with the highest degree of similarity is more than the threshold confidence level, store the discrete value associated with the highest degree of similarity as the biometric content information corresponding to the biometric information of the user.

In some implementations, the output requesting the feedback from the user confirming the configuration of the display output space includes enabling the user to provide an input to the computing device indicating an actual layout of the display output space as presented on the biometric sensing computing device.

In some implementations, the one or more processors are configured to execute the one or more instructions stored in the one or more memories to store the biometric content information in a remote server computing system.

In some implementations, the one or more processors are configured to execute the one or more instructions stored in the one or more memories to automatically store the biometric content information in the remote server computing system in response to the computing device connecting to a network.

In an example embodiment, a computer-implemented method is provided. The computer-implemented method includes obtaining display screen information associated with a display screen of a biometric sensing computing device; obtaining an image including the display screen displaying biometric information associated with a user of the biometric sensing computing device; executing a digital display function based on the display screen information to determine biometric content information from the image corresponding to the biometric information; and storing the biometric content information determined by executing the digital display function.

In some implementations, the method includes obtaining device information associated with the biometric sensing computing device using an image of the biometric sensing computing device; and obtaining the display screen information associated with the display screen of the biometric sensing computing device based on the device information.

In some implementations, the method includes determining the digital display function to be executed based on whether a display output space provided on the display screen includes a discrete display output space and/or a continuous display output space.

In an example embodiment, a non-transitory computer-readable medium which stores instructions that are executable by one or more processors of a computing device is provided. The non-transitory computer-readable medium stores instructions which are executable by one or more processors of the computing device. The instructions include: instructions to cause the one or more processors to: obtain display screen information associated with a display screen of a biometric sensing computing device; obtain an image including the display screen displaying biometric information associated with a user of the biometric sensing computing device; execute a digital display function based on the display screen information to determine biometric content information from the image corresponding to the biometric information; and store the biometric content information determined by executing the digital display function.

The non-transitory computer-readable medium may store additional instructions to execute other aspects and operations of the computing device and computer-implemented method as described herein.

In an example embodiment, a computing device (e.g., a mobile phone, a smartphone, etc.) is provided. The computing device includes one or more memories configured to store one or more instructions and one or more processors configured to execute the one or more instructions stored in the one or more memories to: obtain display screen information associated with a display screen of a biometric sensing computing device, capture, using the one or more cameras, an image of the display screen of the biometric sensing computing device, the display screen displaying biometric information associated with a user, process the image of the display screen by implementing one or more machine-learned models to determine biometric content information from the image corresponding to the biometric information, and store the biometric content information determined by processing the image.

These and other features, aspects, and advantages of various embodiments of the disclosure will become better understood with reference to the following description, drawings, and appended claims. The accompanying drawings, which are incorporated in and constitute a part of this specification, illustrate examples of the disclosure and, together with the description, serve to explain the related principles.

### BRIEF DESCRIPTION OF THE DRAWINGS

Detailed discussion of example embodiments directed to one of ordinary skill in the art is set forth in the specification, which makes reference to the appended drawings, in which:

FIG. 1 is an example system including block diagrams of a user computing device, a server computing system, and a biometric sensing computing device, according to one or more examples of the disclosure;

FIG. 2 is an example illustration of a user computing device capturing an image of a biometric sensing computing device and obtaining biometric content information via the image and a biometric application, according to one or more examples of the disclosure;

FIG. 3 is an example block diagram of a biometric application, according to one or more examples of the disclosure;

FIGS. 4-5 are example illustrations of biometric sensing computing devices and corresponding display output spaces, according to one or more examples of the disclosure;

FIG. 6A-6B are example illustrations of continuous biometric signals, according to one or more examples of the disclosure;

FIGS. 7A-7B are example user interface screens for confirming a configuration of a display output space of a biometric sensing computing device, according to one or more examples of the disclosure; and

FIG. 8 is a flow diagram of an example, non-limiting computer-implemented method according to one or more examples of the disclosure.

### DETAILED DESCRIPTION

Reference now will be made to embodiments of the disclosure, one or more examples of which are illustrated in the drawings. Each example is provided by way of explanation of the disclosure and is not intended to limit the disclosure. In fact, it will be apparent to those skilled in the art that various modifications and variations can be made to the disclosure without departing from the scope or spirit of the disclosure. For instance, features illustrated or described as part of one embodiment can be used with another embodiment to yield a still further embodiment. Thus, it is intended that the disclosure covers such modifications and variations as come within the scope of the appended claims and their equivalents.

Terms used herein are used to describe the example embodiments and are not intended to limit and/or restrict the disclosure. The singular forms “a,” “an” and “the” are intended to include the plural forms as well, unless the context clearly indicates otherwise. In this disclosure, terms such as “including”, “having”, “comprising”, and the like are used to specify features, numbers, steps, operations, elements, components, or combinations thereof, but do not preclude the presence or addition of one or more of the features, elements, steps, operations, elements, components, or combinations thereof.

It will be understood that, although the terms first, second, third, etc., may be used herein to describe various elements, the elements are not limited by these terms. Instead, these terms are used to distinguish one element from another element. For example, without departing from the scope of the disclosure, a first element may be termed as a second element, and a second element may be termed as a first element.

The term “and/or” includes a combination of a plurality of related listed items or any item of the plurality of related listed items. For example, the scope of the expression or phrase “A and/or B” includes the item “A”, the item “B”, and the combination of items “A and B”.

In addition, the scope of the expression or phrase “at least one of A or B” is intended to include all of the following: (1) at least one of A, (2) at least one of B, and (3) at least one of A and at least one of B. Likewise, the scope of the expression or phrase “at least one of A, B, or C” is intended to include all of the following: (1) at least one of A, (2) at least one of B, (3) at least one of C, (4) at least one of A and at least one of B, (5) at least one of A and at least one of C, (6) at least one of B and at least one of C, and (7) at least one of A, at least one of B, and at least one of C.

Example aspects of the disclosure are directed to a computing device, for example, a portable computing device including a smartphone, that can be used to capture an image including biometric information that is displayed on a display screen of a biometric sensing computing device (e.g., a medical device), and to extract the biometric information from the image based on a digital display function that is based on characteristics of the display screen. The biometric information can be stored, for example at a remote computing device (e.g., a server computing system) so that a user can track health information over time which might otherwise be lost or deleted after a user measures their biometric information.

As an example implementation, a pulse oximeter device may measure an amount of oxygen saturation (SpO2) and the value may be displayed on a light emitting display screen of the pulse oximeter device. Some pulse oximeter devices may delete or stop displaying the SpO2 measurement when the user removes their finger from the pulse oximeter device or after some period of time, and not store the measurements for the user to track over time. According to examples of the disclosure a user may capture an image of the display screen via a camera of a computing device (e.g., a smartphone) while the SpO2 value is being displayed. The computing device may include a biometric application that is configured to perform image processing operations with respect to the image to extract the biometric information from the image and store the biometric information (e.g., at a remote server computing device). The image processing operations may include an operation to run perspective correction using homographic principles to obtain an image that would be captured from a camera viewpoint that is orthogonal to the display screen of the pulse oximeter device. The image processing operations may also include an operation of executing a digital display function (also referred to as a dictionary function) that matches the content displayed on the display screen with display possibilities of the display screen which are known from characteristics of the display screen and layouts (e.g., default layouts) of the display screen. A best match with respect to a biometric measurement that is displayed on the display screen and one of the display possibilities for that biometric measurement may correspond to a match with an optimal similarity metric (e.g., a lowest distance metric) and the best match may be selected as biometric content information (a biometric measurement) to be stored (e.g., in a health database).

For example, the biometric content information may be stored in a remote server computing system. In some implementations, the biometric content information may automatically be stored at the remote server computing system in response to the computing device connecting to a network, at a predetermined time or a predetermined interval of time, in response to a next power-on event of the computing device, etc.

In some implementations the biometric information that is displayed on the display screen of the biometric sensing computing device can include biometric information that is in the form of discrete values (e.g., a blood pressure value, a number of steps walked, etc.) and/or continuous values (e.g., an ECG signal, a PPG signal, etc.). Different digital display functions may be applied to biometric information that is in the form of discrete values compared to biometric information that is in the form of continuous values. For example, a first portion (or first region) of a display screen of the biometric sensing computing device can include biometric information that is expressed with a discrete value and a second portion (or second region) of the display screen can include biometric information that is expressed with a continuous value. A first digital display function may be applied to the captured image with respect to the first portion and a second digital display function may be applied to the captured image with respect to the second portion.

For example, when the first portion of the display output space provided on the display screen of the biometric sensing computing device includes the discrete display output space, the computing device may execute a first digital display function to compare the captured image including the biometric information with each of a plurality of possible discrete values which can be output with respect to the first portion of the display output space. The computing device may have a priori knowledge of the possible values which can be displayed in the first portion of the display screen. For example, for a display output space which can display two digits (e.g., for a two-digit biometric value), there may be 100 possible discrete values, for a display output space which can display three digits (e.g., for a three-digit biometric value), there may be 1000 possible discrete values, and so on. The computing device can, during a matching phase, scan through each of the discrete values and determine a similarity metric value for each comparison and select a discrete value among the plurality of possible discrete values that is associated with the highest degree of similarity based on the similarity metric values determined from the comparisons. The best match or comparison can be based on a normalized L2-distance metric, for example. The disclosed method is a much more robust approach compared to a generic optical character recognition approach which can have noisy estimates based on the image recording fidelity, camera sensor shot noise, etc. The round-robin scanning exploits a strong prior on the display screen specifications to constrain the possible display options and lead to a more physically realistic estimate. The computing device can store the discrete value associated with the highest degree of similarity as the biometric content information corresponding to the biometric information of the user.

For example, when the second portion of the display output space provided on the display screen of the biometric sensing computing device includes the continuous display output space (e.g., for displaying an ECG signal, a PPG signal, etc.), the computing device may execute a second digital display function. For example, executing the second digital display function may include the computing device determining a type of biometric signal displayed in the image. For example, classification, regression, pattern recognition algorithms, and the like can be implemented by the computing device to classify or determine the type of biometric signal displayed in the image based on features extracted from the signal displayed in the image (e.g., peak-to-peak distance, number of peaks, etc.). Executing the second digital display function may further include the computing device applying a computer-implemented spline fitting method to reconstruct the biometric signal displayed in the image to obtain a reconstructed biometric signal, based on the type of biometric signal displayed in the image. For example, data points can be extracted from the image of the biometric signal and a spline technique (e.g., B-spline fitting, Bezier curve fitting, etc.) can be applied to fit smooth curves to those points. The computing device can store the reconstructed biometric signal as the biometric content information corresponding to the biometric information of the user.

In some implementations, when the reconstructed biometric signal includes one or more missing data portions, the computing device may apply a computer-implemented spline interpolation method to fully reconstruct the biometric signal, the computer-implemented spline interpolation method being based on the type of biometric signal displayed in the image. For example, camera sensor noise can lead to missing line segments (e.g., from a false negative from aggressive thresholding to overcome noise). In such cases, the computing device may be configured to implement a high-dimensional spline interpolation (e.g., via a quadratic spline method, a cubic spline method, etc.) that can fit the curve in a fairly overparameterized way, to reconstruct the full waveform digitally.

According to examples of the disclosure, the computing device can be configured to receive information about the biometric sensing computing device in order to apply the appropriate digital display function in an automatic or manual fashion. In some implementations, the computing device can obtain device information associated with the biometric sensing computing device based on an input received from a user (e.g., via a user interface or other input device). In some implementations, the computing device can obtain display screen information associated with the display screen of the biometric sensing computing device based on an input received from a user (e.g., via a user interface or other input device).

In some implementations, the computing device can obtain an image of the biometric sensing computing device and predict or determine the model of the biometric sensing computing device, for example, via one or more machine-learned models. Based on the identification of the biometric sensing computing device, the computing device can obtain device information associated with the biometric sensing computing device, including a device part number, a device model number, a device name, a device manufacturer, biometric device capabilities associated with the biometric sensing computing device, physical dimensions and weight, and the like.

The computing device can also obtain display screen information associated with the display screen of the biometric sensing computing device based on the device information. For example, specification information associated with the display screen can be obtained. The display screen information can include a resolution of the display screen, a size of the display screen, color information of the display screen, font information of the display screen, a type of the display screen, layout information of the display screen, tolerance information associated with the display screen, text placement information of the display screen, and the like. The display screen information can be used to determine the appropriate digital display function and for identifying information presented on the display screen in the image, for example, based on known possible configurations of the display screen as indicated from the display screen information. For example, the biometric device capability information can be used to determine the appropriate digital display function(s) that may be applicable for identifying or reproducing the biometric information presented on the display screen of the biometric sensing computing device. For example, the biometric device capabilities associated with the biometric sensing computing device can include one or more capabilities to generate an electrocardiogram, generate a photoplethysmogram, measure heart rate, measure blood pressure, measure oxygen saturation, measure respiration rate, measure body temperature, measure physical activity, measure a sleep metric, measure electrical conductance, and the like. For example, when the biometric sensing computing device is not capable of displaying a continuous biometric signal (e.g., a continuous biometric signal including ECG signals, PPG signals, etc.), the computing device may be configured to ignore or not apply digital display functions that are associated with continuous biometric signals, thereby conserving computing resources of the computing device, and improving the efficiency of the computing device.

According to additional aspects of the disclosure, when a confidence value associated with a best match or a reconstructed biometric signal is less than a threshold confidence level, the computing device may request feedback from a user associated with the biometric sensing computing device, for example, to confirm a configuration of the display output space of the biometric sensing computing device. For example, the computing device may request that the user confirm whether the assumed layout of the display output space used for the digital display function is accurate. If the assumed layout is not correct, the user may provide an input identifying the correct or actual layout of the display output space as presented on the biometric sensing computing device. For example, a user may provide an input to a user interface screen where a representation of the display screen of the biometric sensing computing device is shown, manipulating various display output space regions to match the actual layout or configuration of the display output space regions on the display screen of the biometric sensing computing device. Thereafter, the computing device may re-execute the digital display function based on the updated or corrected configuration of the display output space of the biometric sensing computing device.

Example aspects of the disclosure provide several technical effects, benefits, and/or improvements in computing technology and the technology of computing devices and health monitoring devices. For example, according to one or more examples of the disclosure, biometric measurements can be collected and recorded in an accurate and efficient manner by use of a digital display function which analyzes an image of a display screen of a biometric sensing computing device to obtain biometric content information associated with a user of the biometric sensing computing device. The digital display function is an improvement over existing optical character recognition methods which may rely on a document or image to be of a certain format, have a certain font, etc. In addition, according to one or more examples of the disclosure, biometric measurements can be collected and recorded from a biometric sensing computing device which does not require a wired or wireless connection. For example, the disclosed method can be applied to biometric sensing computing devices which do not have the ability to connect to the internet or other devices.

For example, according to one or more examples of the disclosure, user friction associated with obtaining biometric measurements can also be reduced by providing for the automatic recognition of biometric sensing computing devices, the automatic collection of display screen specifications, thus eliminating or reducing the need for user interaction which prevents or reduces errors from occurring due to human error in manually inputting data. For example, users may be unfamiliar with the types of biometric signals which are being displayed on the biometric sensing computing device and may incorrectly identify or input information about those biometric signals. In addition, users may incorrectly input data due to typographical errors, being illiterate, having poor eyesight, etc. Therefore, aspects of the disclosure provide the technical effect, benefit, and/or improvements in computing technology and the technology of computing devices and health monitoring devices by accurately collecting and recording biometric information associated with a user through reducing the need for user interactions or inputs.

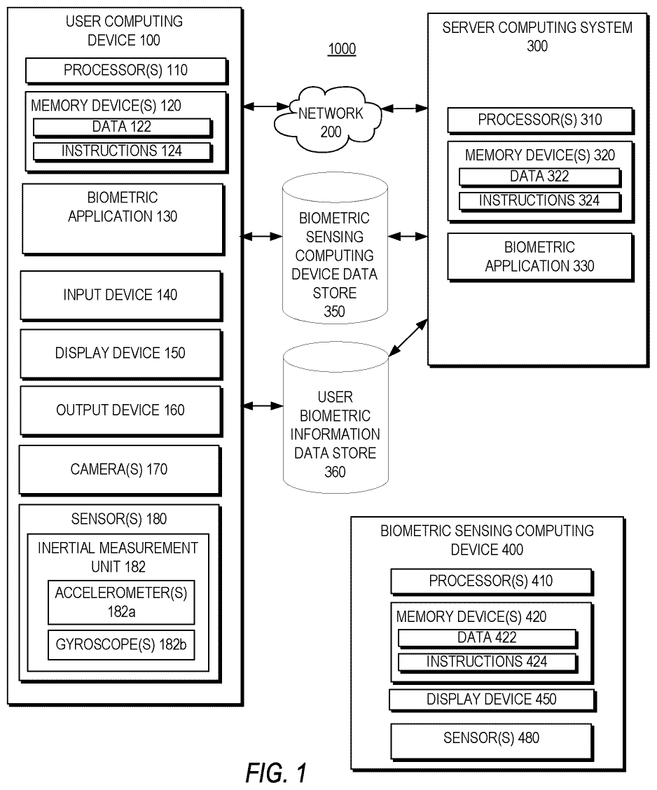

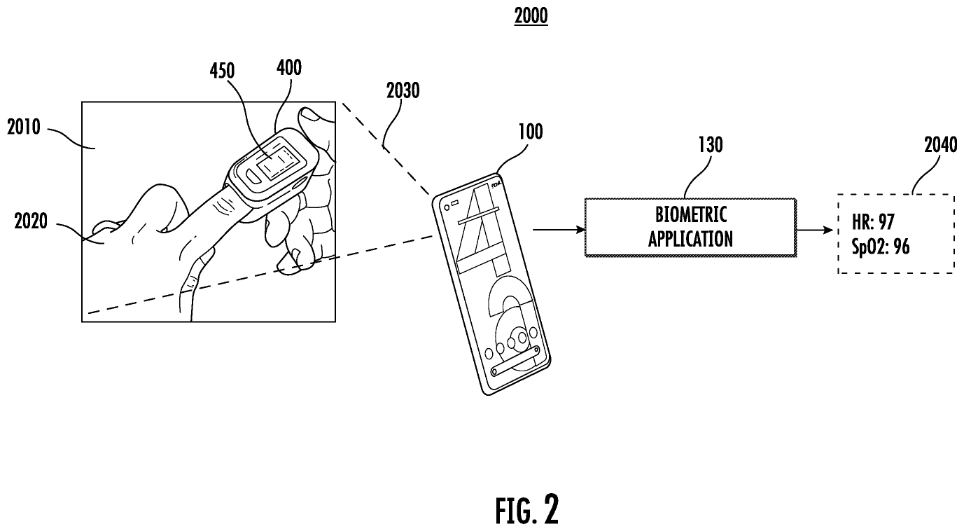

Referring now to the drawings, FIG. 1 illustrates an example system including block diagrams of a user computing device, a server computing system, and a biometric sensing computing device, according to one or more examples of the disclosure. FIG. 2 is an example illustration of a user computing device capturing an image of a biometric sensing computing device and obtaining biometric content information via the image and a biometric application, according to one or more examples of the disclosure.

In FIG. 1, the example system 1000 includes a user computing device 100, a server computing system 300, and a biometric sensing computing device 400. For example, the user computing device 100 and server computing system 300 may be connected with one another over a network 200. For example, the biometric sensing computing device 400 may not be connected to network 200 or be capable of connecting to the user computing device 100 and server computing system 300 (e.g., in a wired or wireless manner). Any communications interfaces suitable for communicating via the network 200 (such as a network interface card) may be utilized as appropriate or desired by the user computing device 100 and server computing system 300.

The user computing device 100 may include a personal computer, a smartphone, a laptop, a tablet computer, and the like. The server computing system 300 may include a server, or a combination of servers (e.g., a web server, application server, etc.) in communication with one another, for example in a distributed fashion. The biometric sensing computing device 400 may include a pulse oximeter, heart rate monitor, blood pressure monitor, electrodermal activity sensor, glucometer, body temperature monitor, sleep tracker, electrocardiogram devices, biometric wearable computing devices (e.g., a biometric smartwatch), and the like. The biometric sensing computing device 400 may be configured to measure various biometrics, including biometrics associated with an ECG, PPG, heart rate, pulse information, BMI, heart rate variability, blood pressure, oxygen saturation, body temperature, sleep quality, physical activities (e.g., number of steps walked), and the like. Further, the biometric sensing computing device 400 may be configured to generate or display information associated with an electrocardiogram, a photoplethysmogram, heart rate, blood pressure, oxygen saturation, respiration rate, body temperature, physical activity, a sleep metric, electrical conductance, and the like.

Referring to FIG. 2, according to some implementations of the disclosure, in the illustrated overview 2000 the user computing device 100 may be configured to capture an image 2010 within a field of view 2030 of the one or more cameras 170 of the user computing device 100. For example, the image 2010 may include a user 2020 wearing the biometric sensing computing device 400 which has a display device 450 that outputs or displays biometric information (e.g., in a digital form with discrete values).

As explained in more detail herein, user computing device 100 may be configured to obtain display screen information associated with the display screen of the biometric sensing computing device 400. For example, the user computing device 100 may be configured to obtain an image of the biometric sensing computing device 400, obtain device information associated with the biometric sensing computing device 400 based on the image of the biometric sensing computing device, and obtain the display screen information associated with the display screen of the biometric sensing computing device based on the device information. For example, the user computing device 100 may be configured to receive information associated with a user input providing device information (e.g., a make and/or model) associated with the biometric sensing computing device 400 and obtain the display screen information associated with the display screen of the biometric sensing computing device 400 based on the device information.

For example, in some implementations the user computing device 100 may identify the biometric sensing computing device 400 (e.g., by make and model type) from the image 2010 by locating the biometric sensing computing device 400 within the field of view 2030 of the one or more cameras 170 of the user computing device 100, extracting a region in the image 2010 which includes the biometric sensing computing device 400, extracting relevant features of the located biometric sensing computing device 400 (e.g., a color, shape, texture, logos, etc.), and classifying and recognizing the biometric sensing computing device 400 (e.g., via a machine-learned model which is trained to predict a make and model of a biometric sensing computing device from an image). Thereafter, specification information (e.g., including display screen information) of the biometric sensing computing device 400 can be obtained by the user computing device 100 (e.g., from biometric sensing computing device data store 350).

Further, the user computing device 100 may be configured to execute a digital display function via biometric application 130 based on the display screen information associated with the display device 450 to determine biometric content information 2040 from the image 2010 corresponding to the biometric information. The biometric content information 2040 can be stored, for example, at the user computing device 100 or remotely at server computing system 300 or user biometric information data store 360 via network 200. As illustrated in FIG. 2, the biometric content information 2040 corresponds to the biometric information that is displayed via the display device 450 in that the digital display function is configured to determine the biometric information from the image 2010 without a user manually inputting the biometric information. Thus, the biometric content information 2040 (e.g., a heart rate of 97 and oxygen saturation of 96%) output by the biometric application 130 should match or correspond to the biometric information as displayed on the display device 450.

For example, the network 200 may include any type of communications network such as a local area network (LAN), wireless local area network (WLAN), wide area network (WAN), personal area network (PAN), virtual private network (VPN), or the like. For example, wireless communication between elements of the examples described herein may be performed via a wireless LAN, Wi-Fi, Bluetooth, ZigBee, Wi-Fi direct (WFD), ultra wideband (UWB), infrared data association (IrDA), Bluetooth low energy (BLE), near field communication (NFC), a radio frequency (RF) signal, and the like. For example, wired communication between elements of the examples described herein may be performed via a pair cable, a coaxial cable, an optical fiber cable, an Ethernet cable, and the like. Communication over the network can use a wide variety of communication protocols (e.g., TCP/IP, HTTP, SMTP, FTP), encodings or formats (e.g., HTML, XML), and/or protection schemes (e.g., VPN, secure HTTP, SSL).

The user computing device 100 may include one or more processors 110, one or more memory devices 120, a biometric application 130, an input device 140, a display device 150, an output device 160, one or more cameras 170, and one or more sensors 180. Each of the components of the user computing device 100 may be operatively connected with one another via a system bus. For example, the system bus may be any of several types of bus structures that may further interconnect to a memory bus (with or without a memory controller), a peripheral bus, and/or a local bus using any of a variety of commercially available bus architectures.

The server computing system 300 may include one or more processors 310, one or more memory devices 320, and a biometric application 330. Each of the features of the server computing system 300 may be operatively connected with one another via a system bus. For example, the system bus may be any of several types of bus structures that may further interconnect to a memory bus (with or without a memory controller), a peripheral bus, and/or a local bus using any of a variety of commercially available bus architectures.

The biometric sensing computing device 400 can include any device that is capable of measuring biometric information of a user. In embodiments described herein, the biometric sensing computing device 400 includes a display device 450 which outputs the biometric information on a display screen. The biometric sensing computing device 400 may include one or more processors 410, one or more memory devices 420, the display device 450, and one or more sensors 480. Each of the features of the biometric sensing computing device 400 may be operatively connected with one another via a system bus. For example, the system bus may be any of several types of bus structures that may further interconnect to a memory bus (with or without a memory controller), a peripheral bus, and/or a local bus using any of a variety of commercially available bus architectures.

For example, the one or more processors 110, 310, 410 can be any suitable processing device that can be included in a user computing device 100, server computing system 300, or biometric sensing computing device 400. For example, such a processor 110, 310, 410 may include one or more of a processor, processor cores, a controller and an arithmetic logic unit, a central processing unit (CPU), a graphics processing unit (GPU), a digital signal processor (DSP), an image processor, a microcomputer, a field programmable array, a programmable logic unit, an application-specific integrated circuit (ASIC), a microprocessor, a microcontroller, etc., and combinations thereof, including any other device capable of responding to and executing instructions in a defined manner. The one or more processors 110, 310, 410 can be a single processor or a plurality of processors that are operatively connected, for example in parallel.

The one or more memory devices 120, 320, 420 can include one or more non-transitory computer-readable storage mediums, such as such as a Read Only Memory (ROM), Programmable Read Only Memory (PROM), Erasable Programmable Read Only Memory (EPROM), and flash memory, a USB drive, a volatile memory device such as a Random Access Memory (RAM), an internal or external hard disk drive (HDD), floppy disks, a blue-ray disk, or optical media such as CD ROM discs and DVDs, and combinations thereof. However, examples of the one or more memory devices 120, 320, 420 are not limited to the above description, and the one or more memory devices 120, 320, 420 may be realized by other various devices and structures as would be understood by those skilled in the art.

For example, the one or more memory devices 120 can store instructions, that when executed, cause the one or more processors 110 to obtain display screen information associated with a display screen of the biometric sensing computing device 400, obtain an image including the display screen, the display screen displaying biometric information associated with a user of the biometric sensing computing device 400, execute a digital display function based on the display screen information to determine biometric content information from the image corresponding to the biometric information, and store the biometric content information determined by executing the digital display function, as described according to examples of the disclosure.

For example, the one or more memory devices 320 can store instructions, that when executed, cause the one or more processors 310 to receive from the user computing device 100 an image including a display screen of the biometric sensing computing device 400, the display screen displaying biometric information associated with a user of the biometric sensing computing device 400, obtain display screen information associated with the display screen of the biometric sensing computing device 400, execute a digital display function based on the display screen information to determine biometric content information from the image corresponding to the biometric information, and store the biometric content information determined by executing the digital display function, as described according to examples of the disclosure.

For example, the one or more memory devices 420 can store instructions, that when executed, cause the one or more processors 410 to detect or obtain biometric information associated with a user via the one or more sensors 480 and to provide for display the biometric information on a display screen of the display device 450, as described according to examples of the disclosure.

The one or more memory devices 120 can also include data 122 and instructions 124 that can be retrieved, manipulated, created, or stored by the one or more processors 110. In some examples, such data can be accessed and used as input to obtain and output the biometric content information associated with a user, as described according to examples of the disclosure. The one or more memory devices 320 can also include data 322 and instructions 324 that can be retrieved, manipulated, created, or stored by the one or more processors 310. In some examples, such data can be accessed and used as input to obtain and output the biometric content information associated with a user, as described according to examples of the disclosure. The one or more memory devices 420 can also include data 422 and instructions 424 that can be retrieved, manipulated, created, or stored by the one or more processors 410. In some examples, such data can be accessed and used as input to obtain and output biometric information associated with a user via the one or more sensors 480 and the display device 450, as described according to examples of the disclosure.

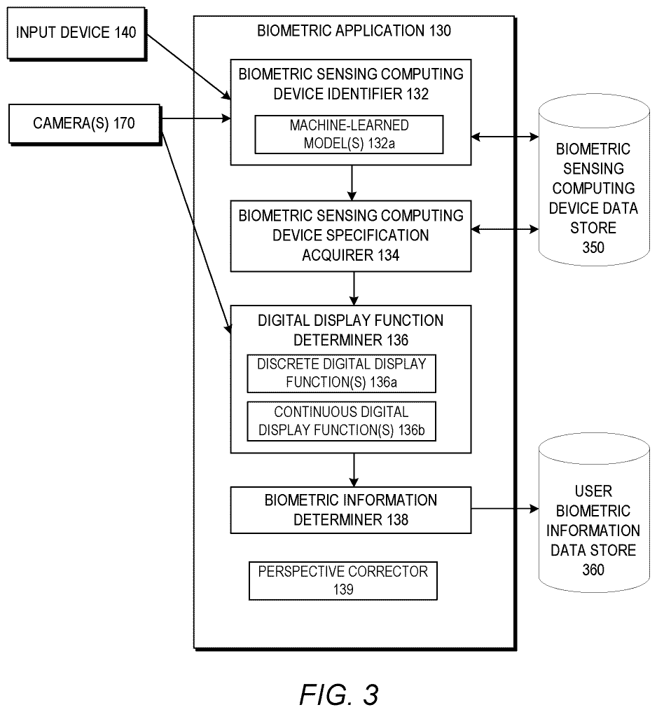

The biometric application 130 can include any biometric application which allows or is capable of determining biometric content information associated with a user based on an image which includes biometric information. As explained with reference to FIG. 3, in some implementations, the biometric application 130 includes a biometric sensing computing device identifier 132, a biometric sensing computing device specification acquirer 134, a digital display function determiner 136, a biometric information determiner 138, and a perspective corrector 139. For example, a user may execute the biometric application 130 by providing an input to the user computing device 100 to determine and store a biometric measurement that is displayed on the biometric sensing computing device 400. The biometric application 330 of the server computing system 300 can also include similar features as the biometric application 130 (e.g., as shown in FIG. 3) which perform similar functions and operations, and therefore will not be repeated for the sake of brevity.

The user computing device 100 may include an input device 140 configured to receive an input from a user and may include, for example, one or more of a keyboard (e.g., a physical keyboard, virtual keyboard, etc.), a mouse, a joystick, a button, a switch, an electronic pen or stylus, a gesture recognition sensor (e.g., to recognize gestures of a user including movements of a body part), an input sound device or voice recognition sensor (e.g., a microphone to receive a voice command), a track ball, a remote controller, a portable (e.g., a cellular or smart) phone, and so on. The input device 140 may also be embodied by a touch-sensitive display device having a touchscreen capability, for example. The input device 140 may be used by a user of the user computing device 100 to provide an input to request to capture an image of the biometric sensing computing device 400, to provide an input to execute the biometric application 130 (see FIG. 3), to transmit biometric content information of the user to the server computing system 300, etc. For example, the input may be a voice input, a touch input, a gesture input, a click via a mouse or remote controller, and so on.

The user computing device 100 may include a display device 150 which presents information viewable by the user, for example on a user interface (e.g., a graphical user interface). For example, the display device 150 may be a non-touch sensitive display. The display device 150 may include a liquid crystal display (LCD), a light emitting diode (LED) display, an organic light emitting diode (OLED) display, active matrix organic light emitting diode (AMOLED), flexible display, 3D display, a plasma display panel (PDP), a cathode ray tube (CRT) display, and the like, for example. However, the disclosure is not limited to these example display devices and may include other types of display devices.

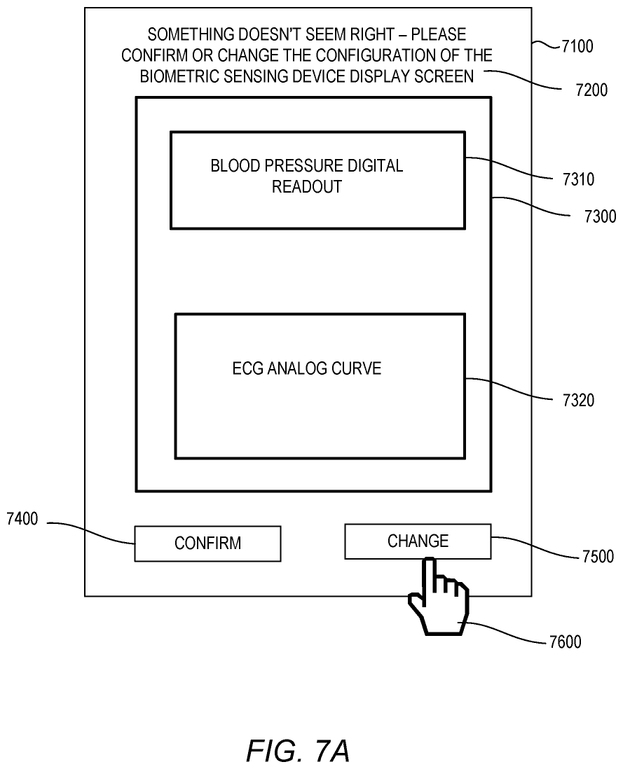

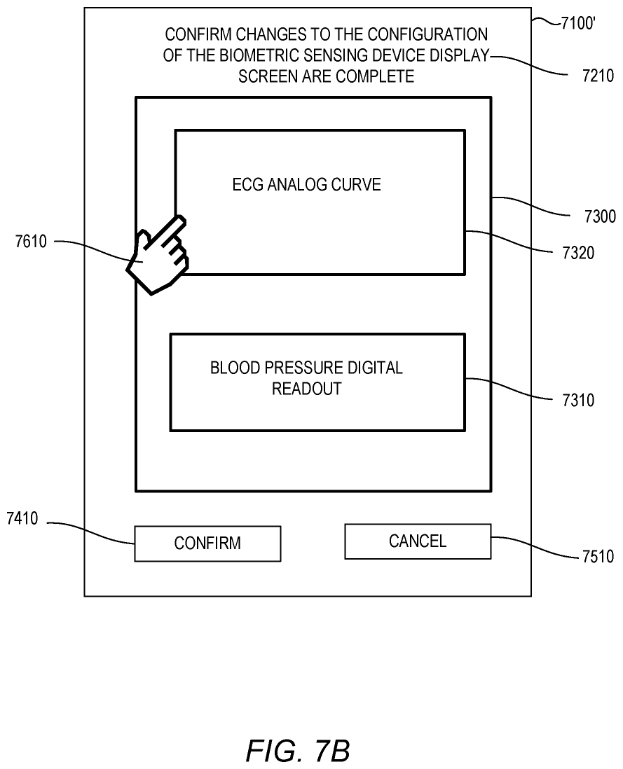

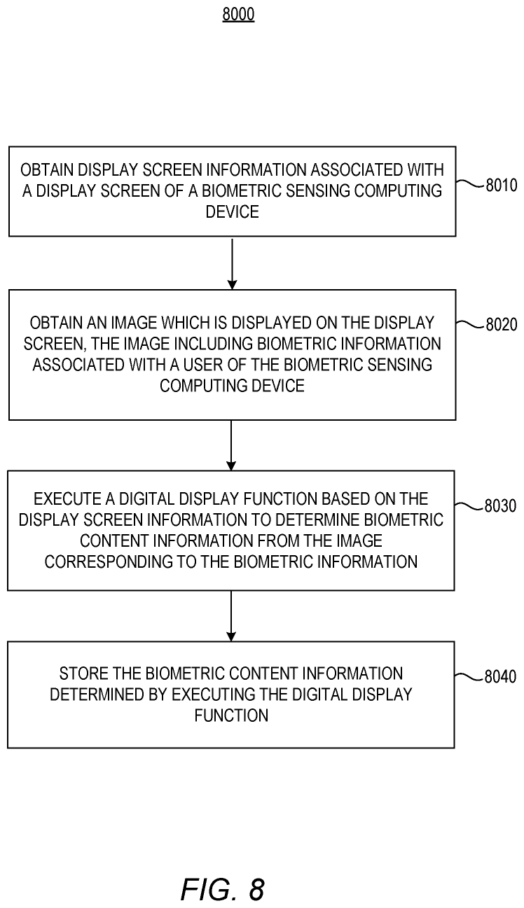

The user computing device 100 may include an output device 160 configured to provide an output to the user and may include, for example, one or more of an audio device (e.g., one or more speakers), a haptic device to provide haptic feedback to a user, a light source (e.g., one or more light sources such as LEDs which provide visual feedback to a user), and the like. For example, in some implementations of the disclosure the user may be guided through a process for providing feedback regarding a configuration of a display screen of the biometric sensing computing device 400 (e.g., see FIGS. 7A-7B), for example, to aid the biometric application 130 in selecting an appropriate digital display function to determine the biometric information displayed on the display screen of the display device 450.

The user computing device 100 may include one or more cameras 170. For example, the one or more cameras 170 may include an imaging sensor (e.g., a complementary metal-oxide-semiconductor (CMOS) or charge-coupled device (CCD)) to capture, detect, or recognize a user's behavior, figure, expression, etc. In some implementations, the one or more cameras 170 may be used to detect or capture an image of the biometric sensing computing device 400, and in particular, an image of the display screen of the display device of the biometric sensing computing device 400.

The user computing device 100 may include one or more sensors 180. For example, the one or more sensors 180 may include an inertial measurement unit 182 which includes one or more accelerometers 182a and/or one or more gyroscopes 182b. The one or more accelerometers 182a may be used to capture motion information with respect to the user computing device 100. The one or more gyroscopes 182b may also be used additionally or alternatively to capture motion information with respect to the user computing device 100. For example, when the user computing device 100 is used to capture an image of the biometric sensing computing device 400, the one or more accelerometers 182a and/or one or more gyroscopes 182b may provide or output information indicating a tilt and/or angle of the user computing device 100 or a tilt and/or angle of the one or more cameras 170 which capture the image. For example, the motion information (e.g., the tilt and/or angle information) may be used by the user computing device 100 to correct a perspective of the image of the biometric sensing computing device 400. For example, the inertial measurement unit 182 may be configured as a six-axis or six-dimensional inertial measurement unit (e.g., a tri-axial accelerometer and a tri-axial gyroscope). The one or more sensors 180 may also include other sensors such as a magnetometer, GPS sensor, proximity sensor, and the like.

The biometric sensing computing device 400 may include a display device 450 which presents information (e.g., biometric information) viewable by the user, for example on a user interface (e.g., a graphical user interface). For example, the display device 450 may be a non-touch sensitive display. The display device 450 may include a liquid crystal display (LCD), a light emitting diode (LED) display, an organic light emitting diode (OLED) display, active matrix organic light emitting diode (AMOLED), flexible display, 3D display, a plasma display panel (PDP), a cathode ray tube (CRT) display, and the like, for example. However, the disclosure is not limited to these example display devices and may include other types of display devices.

The biometric sensing computing device 400 may include one or more sensors 480. For example, the one or more sensors 480 may include any sensors which can be used to measure biometric information of a user. For example, the one or more sensors 480 may include an inertial measurement unit which includes one or more accelerometers and/or one or more gyroscopes, heart rate sensors, blood pressure sensors, ECG sensors, PPG sensors, galvanic skin sensors, blood oxygen sensors, body temperature sensors, respiration rate sensors, and the like. The one or more sensors 480 may also include other sensors such as a magnetometer, GPS sensor, proximity sensor, optical sensors, force sensors, and the like.

Example system 1000 may include a biometric sensing computing device data store 350. In some implementations, biometric sensing computing device data store 350 can represent a single database. In some implementations, the biometric sensing computing device data store 350 represents a plurality of different databases accessible to the user computing device 100 and server computing system 300. In some examples, the biometric sensing computing device data store 350 can include information relating to biometric sensing computing devices. For example, the biometric sensing computing device data store 350 can include images of biometric sensing computing devices that may be used to train a machine-learned model for identifying a biometric sensing computing device. For example, the biometric sensing computing device data store 350 can include device information associated with reach of the biometric sensing computing devices (e.g., a device part number, a device model number, a device name, a device manufacturer, and the like). For example, the biometric sensing computing device data store 350 can include associated QR codes or other bar codes that can be used to identify a biometric sensing computing device. For example, the biometric sensing computing device data store 350 can include information relating to capabilities of each of the biometric sensing computing devices. For example, the biometric device capabilities associated with the biometric sensing computing devices may include a capability to generate an electrocardiogram, generate a photoplethysmogram, measure heart rate, measure blood pressure, measure oxygen saturation, measure respiration rate, measure body temperature, measure physical activity (e.g., a number of steps taken, miles ran, etc.), measure a sleep metric, measure electrical conductance, and the like.

For example, the biometric sensing computing device data store 350 can include specification information about each of the biometric sensing computing devices. For example, the specification information can include tolerance information associated with the biometric sensing computing device. For example, the specification information can include display screen information associated with a display device of the biometric sensing computing device. The display screen information may include a resolution of the display screen, a size of the display screen, color information of the display screen, font information of the display screen, a type of the display screen, layout information of the display screen, tolerance information associated with the display screen, text placement information of the display screen, and the like. For example, default layout information may be included as part of the display screen information such that particular regions of the display screen may be dedicated or configured for displaying particular biometric information in a particular manner.

Example system 1000 may include a user biometric information data store 360. In some implementations, user biometric information data store 360 can represent a single database. In some implementations, the user biometric information data store 360 represents a plurality of different databases accessible to the user computing device 100 and server computing system 300. In some examples, the user biometric information data store 360 can include biometric information of a user or a plurality of users (e.g., a hospital or medical facility database). In some examples, the user biometric information data store 360 can include information regarding one or more user profiles, including a variety of user data such as user preference data, user demographic data, user calendar data, user social network data, user historical health data, and the like. For example, the user biometric information data store 360 can include any biometric information or information associated with the biometric information (e.g., time information associated with the collection of the biometric information, location information associated with the collection of the biometric information, etc.). The user biometric information data store 360 can include the image which was captured by the user computing device 100 to determine the biometric content information, as described herein. The user biometric information and associated information may be associated with a user account.

The user biometric information data store 360 is provided to illustrate potential data that could be analyzed or stored, in some embodiments, by the user computing device 100 and/or server computing system 300 to maintain a record of biometric information associated with a user, for example. However, such user data may not be collected, used, or analyzed unless the user has consented after being informed of what data is collected and how such data is used. Further, in some embodiments, the user can be provided with a tool (e.g., in a biometric application or via a user account) to revoke or modify the scope of permissions. In addition, certain information or data can be treated in one or more ways before it is stored or used, so that personally identifiable information is removed or stored in an encrypted fashion. Thus, particular user information stored in the user biometric information data store 360 may or may not be accessible to the user computing device 100 and/or server computing system 300 based on permissions given by the user, or such data may not be stored in the user biometric information data store 360 at all.

Referring to FIG. 3, an example block diagram of a biometric application is shown, according to one or more examples of the disclosure. FIG. 3 illustrates that the biometric application 130 includes a biometric sensing computing device identifier 132, a biometric sensing computing device specification acquirer 134, a digital display function determiner 136, a biometric information determiner 138, and a perspective corrector 139. However, the biometric application 130 may include fewer or more features than that shown in FIG. 3. For example, any of the features or operations of the components of biometric application 130 may be provided separately from the biometric application 130. For example, some operations (such as the identification of the biometric sensing computing device by the biometric sensing computing device identifier 132 and/or the acquiring of the display screen information by the biometric sensing computing device specification acquirer 134) may instead be performed by the server computing system 300 (e.g., via biometric application 330).

Operations of the biometric application 130 will now be described in more detail with reference to FIGS. 3 through 8.

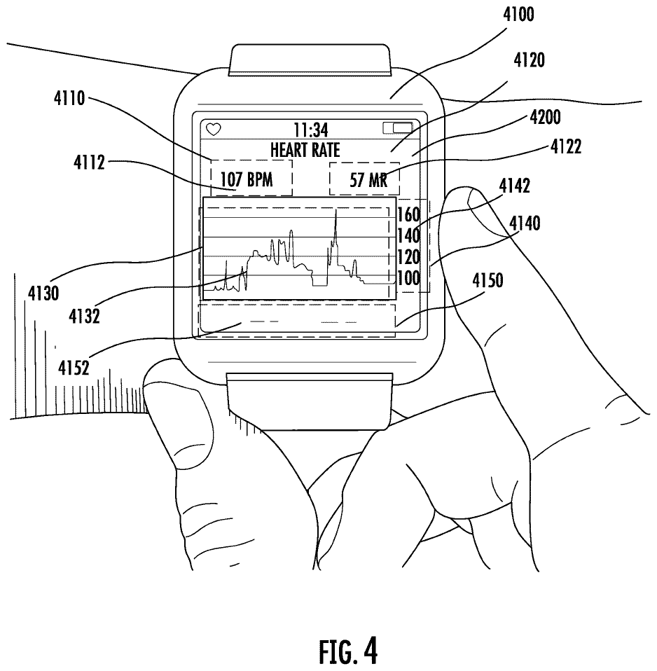

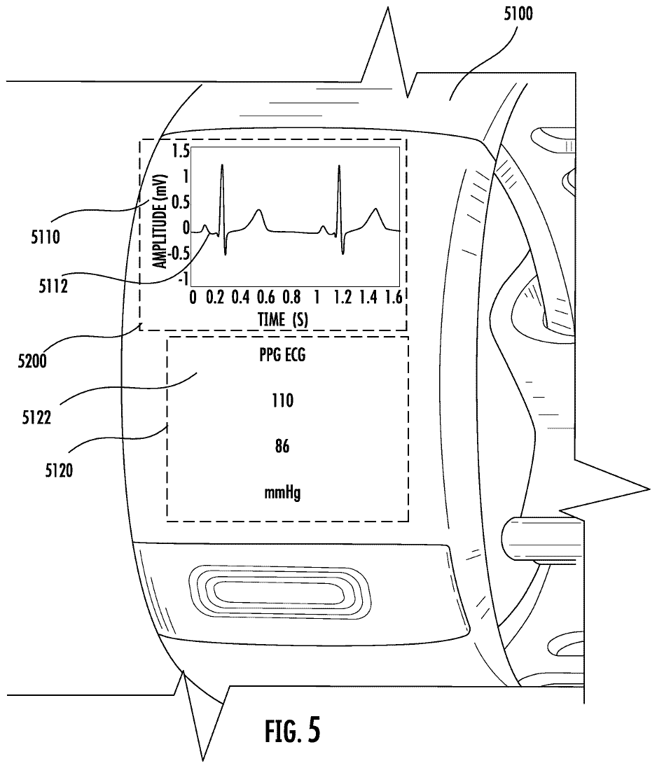

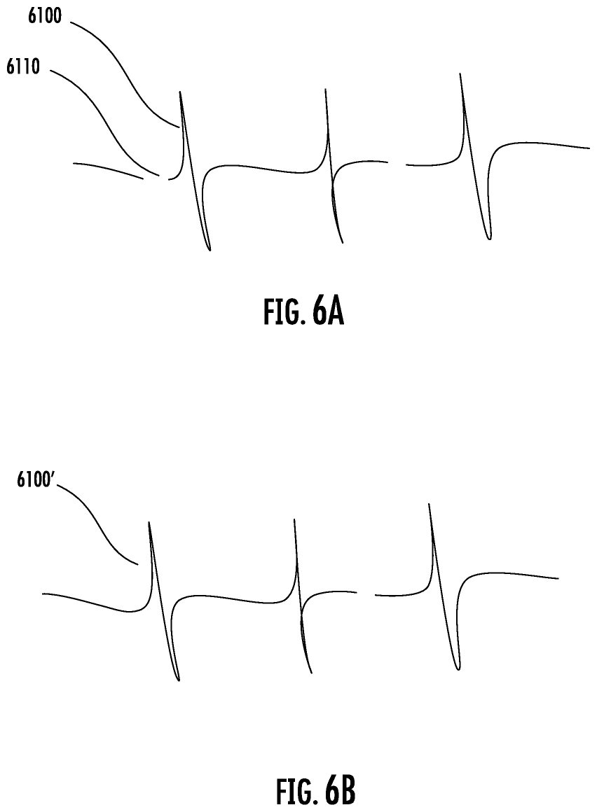

FIGS. 4-5 are example illustrations of biometric sensing computing devices and corresponding display output spaces, according to one or more examples of the disclosure. FIG. 6A-6B are example illustrations of continuous biometric signals, according to one or more examples of the disclosure. FIGS. 7A-7B are example user interface screens for confirming a configuration of a display output space of a biometric sensing computing device, according to one or more examples of the disclosure. FIG. 8 illustrates an example flow diagram of a non-limiting computer-implemented method for determining and storing biometric information of a user, according to one or more examples of the disclosure.

The flow diagram of FIG. 8 illustrates a method 8000 for determining and storing biometric information of a user. Although shown in a particular sequence or order, unless otherwise specified, the order of the processes can be modified. Thus, the illustrated embodiments should be understood only as examples, and the illustrated processes can be performed in a different order, and some processes can be performed in parallel. Additionally, one or more processes can be omitted in various embodiments. Thus, not all processes are required in every embodiment. Other process flows are possible.

At 8010, the method 8000 includes obtaining display screen information associated with a display screen of a biometric sensing computing device. For example, the biometric sensing computing device identifier 132 can identify the biometric sensing computing device 400 and the biometric sensing computing device specification acquirer 134 can obtain display screen information associated with the display screen of the biometric sensing computing device 400 based on the known identity of the biometric sensing computing device 400 (e.g., via a mapping relationship).

In some implementations, the biometric sensing computing device identifier 132 can identify the biometric sensing computing device 400 based on a user input received via the input device 140 where the user provides information about the biometric sensing computing device 400 (e.g., a make and/or model of the biometric sensing computing device 400). In some implementations, the biometric sensing computing device identifier 132 can identify the biometric sensing computing device 400 based on an image of the biometric sensing computing device 400 captured by the one or more cameras 170. In some implementations, the biometric sensing computing device identifier 132 can identify the biometric sensing computing device 400 based on a combination of inputs (e.g., from the input device 140 and one or more cameras 170).

In some implementations, the biometric sensing computing device identifier 132 may be configured to identify the biometric sensing computing device 400 based on a user input received via the input device 140 providing identifying information about the biometric sensing computing device 400 (e.g., a make and/or model of the biometric sensing computing device 400). In some implementations, the biometric sensing computing device 400 may store information about one or more biometric sensing computing devices 400. The biometric sensing computing device identifier 132 may be configured to identify the biometric sensing computing device 400 based on a user input received via the input device 140 selecting one of the one or more biometric sensing computing devices 400 (e.g., via a pull-down menu, via a voice input, touch-input, or other user interface method) which may be displayed to the user via display device 150 to choose from. Device information about the one or more biometric sensing computing devices 400 may include a device part number, a device model number, a device name, a device manufacturer, biometric device capabilities associated with the biometric sensing computing device 400, and the like.

In some implementations, the biometric sensing computing device identifier 132 may be configured to identify the biometric sensing computing device 400 by capturing an image of a barcode or QR code that is disposed on the biometric sensing computing device 400. For example, the barcode or QR code may include encoded data with identifying information about the biometric sensing computing device 400 or information (e.g., a URL) which can be used to obtain identifying information about the biometric sensing computing device 400 from another source (e.g., from biometric sensing computing device data store 350).

In some implementations, the biometric sensing computing device identifier 132 may be configured to identify the biometric sensing computing device 400 via one or more machine-learned models 132a. For example, the biometric sensing computing device identifier 132 may be configured to locate the biometric sensing computing device 400 within the image captured by the one or more cameras 170, for example by use of one or more object detection algorithms including a region-based Convolutional Neural Network (R-CNN) object detection algorithm, a You Only Look Once (YOLO) object detection algorithm, or a single shot multibox detector (SSD) object detection algorithm, and the like. For example, the biometric sensing computing device identifier 132 may be configured to locate the biometric sensing computing device 400 within the image captured by the one or more cameras 170, for example by use of one or more object detection algorithms including a region-based Convolutional Neural Network (R-CNN) object detection algorithm, a You Only Look Once (YOLO) object detection algorithm, or a single shot multibox detector (SSD) object detection algorithm, and the like. For example, the biometric sensing computing device identifier 132 may be configured to extract a region in the image which includes the biometric sensing computing device 400 and extract relevant features of the located biometric sensing computing device 400 (e.g., a color, shape, texture, logos, labels, etc.). For example, the biometric sensing computing device identifier 132 may be configured to implement the one or more machine-learned models 132a (e.g., a support vector machine, deep neural network, etc.) to analyze the extracted features from the image or region from the image including the biometric sensing computing device 400 and compare the features with learned patterns to determine or predict the most likely identity (e.g., make and model) of the biometric sensing computing device 400. In some implementations, the one or more machine-learned models 132a may be configured to classify the type of biometric sensing computing device according to the capabilities of the biometric sensing computing device 400 to measure certain biometric information and/or according to the capabilities of the biometric sensing computing device 400 to display certain biometric information. As described herein, device information about the biometric sensing computing devices (e.g., a device part number, a device model number, a device name, a device manufacturer, biometric device capabilities associated with the biometric sensing computing device 400, and the like) may be stored at biometric sensing computing device data store 350. Furthermore, training data, images of biometric sensing computing devices, feature information, pattern information, and the like may also be stored at biometric sensing computing device data store 350 for reference by the one or more machine-learned models 132a.

Based on the identity of the biometric sensing computing device 400, specification information (e.g., tolerance information, display screen information, size information, etc.) of the biometric sensing computing device 400 can be obtained by the biometric sensing computing device specification acquirer 134. For example, the specification information may also be obtained from biometric sensing computing device data store 350. The display screen information may include a resolution of the display screen, a size of the display screen, color information of the display screen, font information of the display screen, a type of the display screen, layout information of the display screen, tolerance information associated with the display screen, text placement information of the display screen, and the like. For example, default layout information may be included as part of the display screen information such that particular regions of the display screen may be dedicated or configured for displaying particular biometric information in a particular manner. Biometric device capabilities associated with the biometric sensing computing device 400 may also be stored at biometric sensing computing device data store 350. For example, the biometric device capabilities associated with the biometric sensing computing device 400 may include capabilities to: generate an electrocardiogram, generate a photoplethysmogram, measure heart rate, measure blood pressure, measure oxygen saturation, measure respiration rate, measure body temperature, measure physical activity, measure a sleep metric, measure electrical conductance, and the like.

At 8020, the method 8000 includes obtaining an image including the display screen displaying biometric information associated with a user of the biometric sensing computing device. For example, the biometric sensing computing device identifier 132 can obtain the image via the one or more cameras 170 (e.g., when a user captures an image of the biometric sensing computing device 400 while biometric information is displayed on the display screen of the display device 450).

In some implementations, the image including the display screen displaying the biometric information associated with the user of the biometric sensing computing device may also be the same image that is used by the biometric sensing computing device identifier 132 to identify the biometric sensing computing device 400. In some implementations, the image including the display screen may be different from an image used by the biometric sensing computing device identifier 132 to identify the biometric sensing computing device 400, if the biometric sensing computing device identifier 132 identifies the biometric sensing computing device 400 using an image.

At 8030, the method 8000 includes executing a digital display function based on the display screen information to determine biometric content information from the image corresponding to the biometric information. For example, the digital display function determiner 136 can identify and implement one or more appropriate digital display functions to execute with respect to the image to determine the biometric content information from the image corresponding to the biometric information which is displayed in the image. In some implementations, the digital display function determiner 136 can identify and implement one or more appropriate digital display functions based on the display screen information, and in particular, based on whether a display output space provided on the display screen includes a discrete display output space and/or a continuous display output space. The display output space may include a region or plurality of regions of the display screen in which information (e.g., biometric information) can be displayed on the display screen by the display device 450.

For example, a particular biometric sensing computing device 400 may have a default layout, or a limited number of possible display layouts, by which biometric information may be presented on the display screen. The default layout and/or possible display layouts of a particular biometric sensing computing device 400 may be known by the user computing device 100 in advance, for example, based on the obtained display screen information and/or device information. This information may be used to select one or more appropriate digital display functions by segmenting the display screen into various display output spaces (e.g., according to certain biometric information presented therein, according to a type of biometric information presented therein, etc.).

Finally, at 8040, the method 8000 includes storing the biometric content information determined by executing the one or more digital display functions. For example, the biometric information determiner 138 may be configured to store discrete values associated with the highest degree of similarity as the biometric content information corresponding to the biometric information of the user. For example, the biometric information determiner 138 may be configured to store continuous values and/or a reconstructed biometric signal associated with continuous values, as the biometric content information corresponding to the biometric information of the user.

Though not explicitly shown as an operation of the method 8000, in some implementations the method 8000 may also include an operation of correcting a perspective of an image of the biometric sensing computing device 400 captured by the one or more cameras 170. For example, perspective corrector 139 may be configured to correct a perspective of the image of the biometric sensing computing device 400 used to identify the biometric sensing computing device 400 and to correct a perspective of the image of the biometric sensing computing device 400 which includes the display screen displaying the biometric information associated with the user of the biometric sensing computing device. For example, the perspective corrector 139 may be configured to correct a perspective of the image of the biometric sensing computing device 400 which includes the display screen displaying the biometric information associated with the user of the biometric sensing computing device, before executing the digital display function. The perspective corrector 139 may be configured to perform image processing operations including using homographic principles to obtain an image that would be captured from a camera viewpoint that is orthogonal to the display screen of the biometric sensing computing device 400. For example, a homography matrix may be calculated based on a correspondence between image points and real-world points (e.g., via direct linear transform, least squares, or random sample consensus methods). Information relating to an angle at which the image was captured by the one or more cameras 170 may also be utilized. For example, each pixel in the perspective-corrected image may be determined by the transformation of its corresponding position in the original image based on the homography matrix. As mentioned above, in some implementations the selection of a digital display function and the execution of the digital display function with respect to the image may be based on the perspective corrected image rather than the original captured image. Likewise, in some implementations the identity of the biometric sensing computing device 400 may be based on the perspective corrected image rather than the original captured image. Thus, the perspective corrector 139 may receive as an input a captured image from the one or more cameras 170 and provide as an output a perspective corrected image to any of the biometric sensing computing device identifier 132, the biometric sensing computing device specification acquirer, the digital display function determiner 136, or the biometric information determiner 138.

Referring now to FIG. 4, an illustration of a biometric sensing computing device and corresponding display output spaces is provided. For example, the biometric sensing computing device 4100 of FIG. 4 is a wearable computing device (e.g., a smartwatch). In the example of FIG. 4, the display screen 4200 may be segmented into a first display output space 4110, a second display output space 4120, a third display output space 4130, a fourth display output space 4140, and a fifth display output space 4150.

The first display output space 4110 may be associated with discrete values 4112 including three digits each having ten possible values for a possible 1000 different discrete values and be associated with a biometric measurement of beats per minute. The second display output space 4120 may be associated with discrete values 4122 including two or three digits each having ten possible values for a possible 100 or 1000 different discrete values and be associated with a biometric measurement of a resting heart rate. The third display output space 4130 may be associated with continuous values that can have a predefined range of values (e.g., based on unit values along the x and y axes) for a biometric signal 4132, and be associated with a biometric measurement of beats per minute over a certain duration of time. The fourth display output space 4140 may be associated with discrete values 4142 that correspond to possible values of the amplitude of the biometric signal (e.g., along the y-axis), where the possible values may be limited to a predefined range of values (e.g., from 40 to 200). The fifth display output space 4150 may be associated with discrete values 4152 that correspond to possible values of the minimum and maximum values of the amplitude of the biometric signal 4132, where the possible values may include two or three digits each having ten possible values for a possible 100 or 1000 different discrete values and be associated with a biometric measurement of beats per minute. The display screen 4200 may include more or fewer display output spaces described herein. For example, other information including units of measurement, time information, location information, etc., may also be determined from the image and may be used to limit or weight possible values or matches, or to determine a confidence level with respect to a calculated similarity metric value.

For example, when a first portion of a display output space provided on the display screen includes a discrete display output space, the digital display function determiner 136 may be configured to select and execute a first digital display function (e.g., one or more discrete digital display functions 136a) with respect to the first portion. For example, the digital display function determiner 136 may be configured to select and execute a first digital display function to: compare the image including the biometric information with each of a plurality of possible discrete values which can be output with respect to the first portion of the display output space, to thereby determine a similarity metric value for each comparison, select a discrete value among the plurality of possible discrete values that is associated with a highest degree of similarity based on the similarity metric values determined from the comparisons, and store the discrete value associated with the highest degree of similarity as the biometric content information corresponding to the biometric information of the user. For example, the similarity metric may include a Euclidean or L2-metric distance value, a cosine similarity value, and the like.

By way of example, based on the known display screen information of the biometric sensing computing device 4100, the first digital display function implemented by the biometric information determiner 138 may recognize that a first portion (e.g., an upper left portion of the display screen 4200 corresponding to first display output space 4110) may have 1000 possible discrete values. The biometric information determiner 138 may be configured to compare the image (e.g., the first portion) including the biometric information with each of a plurality of possible discrete values which can be output with respect to the first portion of the display output space, to thereby determine a similarity metric value for each comparison. Therefore, a similarity metric may be obtained for each of the 1000 comparisons in the example corresponding to first display output space 4110. The biometric information determiner 138 may be configured to select a discrete value among the plurality of possible discrete values that is associated with the highest degree of similarity based on the similarity metric values determined from the comparisons. For example, other information including units of measurement, time information, location information, font information of the display output space or display screen, size information of the display output space or display screen, resolution information of the display output space or display screen, color information of the display output space or display screen, layout information of the display output space or display screen, tolerance information associated with the display output space or display screen, text placement information of the display output space or display screen, etc., may also be determined by the biometric information determiner 138 to limit or weight possible values or matches, or to determine a confidence level with respect to a calculated similarity metric value, when comparing discrete value information in a display output space with candidate discrete values.

For example, the biometric information determiner 138 may be configured to select the discrete value if a confidence level associated with the similarity metric value is greater than a threshold confidence level. In some implementations, if the confidence level associated with the similarity metric value is not greater than the threshold confidence level, biometric information determiner 138 may be configured to request feedback from a user confirming a configuration or layout of the biometric sensing computing device 400, as will be explained with respect to FIGS. 7A-7B.

For example, the biometric information determiner 138 may be configured to store the biometric content information determined by executing the digital display function (e.g., the first digital display function). For example, the biometric information determiner 138 may be configured to store the discrete value associated with the highest degree of similarity as the biometric content information corresponding to the biometric information of the user displayed in the first portion (e.g., first display output space 4110). For example, the biometric information determiner 138 may be configured to store the discrete value associated with the highest degree of similarity as the biometric content information corresponding to the biometric information of the user displayed in the first portion. For example, the biometric information determiner 138 may be configured to store the biometric content information in the one or more memory devices 320 of the server computing system 300 or in user biometric information data store 360, etc. For example, the biometric information determiner 138 may be configured to automatically store the biometric content information in the one or more memory devices 320 of the server computing system 300 or in user biometric information data store 360, in response to the user computing device 100 connecting to network 200, in response to a next power-on event of the user computing device 100 after determining the biometric content information, at a preset time, etc.

Referring now to FIG. 5, an illustration of a biometric sensing computing device and corresponding display output spaces is provided. For example, the biometric sensing computing device 5100 of FIG. 5 is a wearable computing device (e.g., a smartwatch). In the example of FIG. 5, the display screen 5200 may be segmented into a first display output space 5110 and a second display output space 5120.

The first display output space 5110 may be associated with continuous values that can have a predefined range of values (e.g., based on unit values along the x and y axes) for a biometric signal 5112, and be associated with biometric measurements of an ECG signal over a certain duration of time. The second display output space 5120 may be associated with discrete values 5122 including two or three digits each having ten possible values for a possible 100 or 1000 different discrete values and be associated with biometric measurements relating to blood pressure including a systolic pressure and a diastolic pressure. The display screen 5200 may include more or fewer display output spaces than those described herein.

For example, other information including units of measurement, time information, location information, font information of the display output space or display screen, size information of the display output space or display screen, resolution information of the display output space or display screen, color information of the display output space or display screen, layout information of the display output space or display screen, tolerance information associated with the display output space or display screen, text placement information of the display output space or display screen, etc., may also be determined by the biometric information determiner 138 to limit or weight possible values or matches, or to determine a confidence level with respect to a calculated similarity metric value, when comparing discrete value information in a display output space with candidate discrete values.

For example, when a second portion of the display output space provided on the display screen includes the continuous display output space, the digital display function determiner 136 may be configured to select and execute a second digital display function (e.g., one or more continuous digital display functions 136b) with respect to the second portion. For example, the digital display function determiner 136 may be configured to select and execute a second digital display function with respect to the second portion to: determine a type of biometric signal displayed in the image, apply a computer-implemented spline fitting method to reconstruct the biometric signal displayed in the image to obtain a reconstructed biometric signal, based on the type of biometric signal displayed in the image, and store the reconstructed biometric signal as the biometric content information corresponding to the biometric information of the user.

By way of example, based on the known display screen information of the biometric sensing computing device 5100, the second digital display function implemented by the biometric information determiner 138 may recognize that a second portion (e.g., an upper portion of the display screen 5200 corresponding to first display output space 5110) may have continuous values. The biometric information determiner 138 may be configured to determine a type of biometric signal displayed in the image. To determine a type of biometric signal displayed in the image, the biometric information determiner 138 may consider the possible biometric signals (e.g., BCG signals, PPG signals, ECG signals, etc.) that can be output by the biometric sensing computing device 400 based on the device information associated with the biometric sensing computing device 400. The biometric information determiner 138 may be configured to analyze and extract features from the biometric signal (e.g., peak amplitudes, time between adjacent peaks, labels, units of measurement, etc.) and compare the extracted features against a reference database or known templates of the biometric signal (e.g., via matching algorithms, similarity metrics, statistical models (regression), etc. Based on the analysis and comparison results, biometric information determiner 138 may be configured to classify the biometric signal (e.g., as an ECG signal, a PPG signal, etc.) and provide an output indicating the type of biometric signal displayed in the upper portion of the display screen 5200.

For example, the biometric information determiner 138 may be configured to apply or execute a computer-implemented spline fitting method (e.g., via one or more of the one or more continuous digital display functions 136b) to reconstruct the biometric signal 5112 displayed in the image to obtain a reconstructed biometric signal, based on the type of biometric signal displayed in the image. For example, data points can be extracted from the image of the biometric signal by the biometric information determiner 138 and a spline technique (e.g., B-spline fitting, Bezier curve fitting, etc.) can be applied by the biometric information determiner 138 to fit smooth curves to those points, for example, based on the biometric signal. In some implementations, if the confidence level associated with the reconstructed biometric signal is not greater than a threshold confidence level, biometric information determiner 138 may be configured to request feedback from a user confirming a configuration or layout of the biometric sensing computing device 400, as will be explained with respect to FIGS. 7A-7B.

For example, other information including units of measurement, time information, location information, font information of the display output space or display screen, size information of the display output space or display screen, resolution information of the display output space or display screen, color information of the display output space or display screen, layout information of the display output space or display screen, tolerance information associated with the display output space or display screen, text or graph placement information of the display output space or display screen, characteristics of the biometric signal (e.g., expected positioning of peaks, etc.), and the like, may also be determined and utilized by the biometric information determiner 138 to limit or weight possible values for data points in fitting the curve, or to determine a confidence level with respect to a calculated data point when fitting the curve, when reconstructing the biometric signal.

For example, the biometric information determiner 138 may be configured to store the biometric content information determined by executing the digital display function (e.g., the second digital display function). For example, the biometric information determiner 138 may be configured to store the reconstructed biometric signal associated with the highest degree of similarity as the biometric content information corresponding to the biometric information of the user displayed in the first portion (e.g., first display output space 5110).

For example, the biometric information determiner 138 may be configured to store the biometric content information in the one or more memory devices 320 of the server computing system 300 or in user biometric information data store 360, etc. For example, the biometric information determiner 138 may be configured to automatically store the biometric content information in the one or more memory devices 320 of the server computing system 300 or in user biometric information data store 360, in response to the user computing device 100 connecting to network 200, in response to a next power-on event of the user computing device 100 after determining the biometric content information, at a preset time, etc.

The biometric information determiner 138 may be configured to, when the reconstructed biometric signal includes one or more missing data portions, apply a computer-implemented spline interpolation method to fully reconstruct the biometric signal. Referring to FIG. 6A, biometric signal 6100 includes missing data portions including missing data portion 6110. For example, the computer-implemented spline interpolation method may be based on the type of biometric signal displayed in the image. For example, camera sensor noise can lead to missing line segments (e.g., from a false negative from aggressive thresholding to overcome noise). In such cases, the biometric information determiner 138 may be configured to implement a high-dimensional spline interpolation (e.g., via a quadratic spline method, a cubic spline method, etc.) that can fit the curve in a fairly overparameterized way, to reconstruct the full waveform digitally as shown by the fully reconstructed biometric signal 6100′ in FIG. 6B.

As discussed above, when a confidence value associated with a best match or a reconstructed biometric signal is less than a threshold confidence level, the computing device may request feedback from a user associated with the biometric sensing computing device, for example, to confirm a configuration of the display output space of the biometric sensing computing device. For example, when a confidence level associated with a discrete value associated with the highest degree of similarity is less than a threshold confidence level, the user computing device 100 may be configured to provide an output requesting feedback from the user confirming a configuration of a display output space. For example, when a confidence level associated with a continuous value or a curve generated based on continuous values is less than a threshold confidence level, the user computing device 100 may be configured to provide an output requesting feedback from the user confirming a configuration of a display output space or a type of biometric signal. When the confidence level associated with a discrete value associated with the highest degree of similarity is more than the threshold confidence level, the user computing device 100 may be configured to store the discrete value associated with the highest degree of similarity as the biometric content information corresponding to the biometric information of the user.

FIGS. 7A-7B are example user interface screens for confirming a configuration of a display output space of a biometric sensing computing device, according to one or more examples of the disclosure.

In FIG. 7A, an example user interface screen 7100 may be displayed on the user computing device 100 indicating that the biometric information determiner 138 is not confident about the determined biometric content information corresponding to the biometric information displayed in the image. For example, a message 7200 may be displayed requesting feedback from the user regarding a configuration of the replicated display screen 7300 of the biometric sensing computing device. In addition, or alternatively, other output methods may be utilized by the output device 160 (e.g., audio, haptic, etc.) to request feedback from the user. Thus, the output requesting the feedback from the user confirming the configuration of the display output space includes enabling the user to provide an input to the user computing device 100 indicating or confirming an actual layout of the display output space as presented on the biometric sensing computing device 400. The user interface screen 7100 may provide a replicated display screen 7300 of the biometric sensing computing device 400 having an assumed or default configuration of display output spaces (e.g., first display output space 7310 and second display output space 7320) presented on the display screen of the biometric sensing computing device 400. For example, the user 7600 may provide an indication confirming the configuration (e.g., via user interface element 7400) or provide an indication that the configuration is incorrect and needs to be changed (e.g., via user interface element 7500).

In FIG. 7B, an example user interface screen 7100′ may be displayed on the user computing device 100 in response to the user providing an indication that the configuration is incorrect and needs to be changed (e.g., via user interface element 7500 in FIG. 7A). For example, as indicated in FIG. 7B, the user 7610 may provide one or more inputs to indicate the correct configuration of the display output spaces provided in the replicated display screen 7300 of the biometric sensing device. For example, in FIG. 7B the first display output space 7310 has switched positions with the second display output space 7320 (e.g., by a user providing an input moving the first display output space 7310 and second display output space 7320).

For example, a message 7210 may be displayed requesting feedback from the user to confirm the changes to the configuration of the replicated display screen 7300 of the biometric sensing computing device. For example, the user 7610 may provide an indication confirming the changes to the configuration (e.g., via user interface element 7410) or provide an indication cancelling the changes to the configuration (e.g., via user interface element 7510). In response to a selection confirming the changes (e.g., via user interface element 7410) updating or modifying the replicated display screen 7300, the biometric information determiner 138 may re-execute the one or more digital display functions based on the feedback received from the user, utilizing the updated or modified configuration of the replicated display screen.

User interface screens other than those described with respect to FIGS. 7A and 7B may be provided to confirm information utilized by the biometric information determiner 138. For example, a user interface screen may be presented to confirm an assumed or default type of biometric signal when a confidence level associated with a continuous value or a curve generated by the biometric information determiner 138 based on continuous values is less than a threshold confidence level. The biometric information determiner 138 may re-execute the one or more digital display functions based on the feedback received from the user, utilizing an updated or modified type of biometric signal.

Aspects of the disclosure have been described in view of the biometric application 130 provided in the user computing device 100 with respect to FIGS. 3 through 8. However, each of those aspects can also be applied to the biometric application 330 provided in the server computing system 300, and thus functions and operations of the biometric application 130 may also be applied and carried out by the biometric application 330 in a similar fashion, but will not be described again for the sake of brevity.

In some implementations, the digital display function may be implemented via one or more machine-learned models (e.g., predictive machine-learned models, generative machine-learned models, etc.) which are trained to receive as an input an image of a display screen of a biometric computing device and to output biometric content information that corresponds to the biometric information which is displayed on the display screen. For example, the user computing device 100 may include one or more cameras, one or more memories configured to store one or more instructions, and one or more processors configured to execute the one or more instructions stored in the one or more memories to: obtain display screen information associated with a display screen of a biometric sensing computing device, capture, using the one or more cameras, an image of the display screen of the biometric sensing computing device, the display screen displaying biometric information associated with a user, process the image of the display screen by implementing one or more machine-learned models to determine biometric content information from the image corresponding to the biometric information, and store the biometric content information determined by processing the image. The one or more processors may also be configured to execute the one or more instructions stored in the one or more memories to: implement one or more machine-learned models to identify the biometric sensing computing device, using an image of the biometric sensing computing device captured by the one or more cameras, obtain device information associated with the biometric sensing computing device using based on the biometric sensing computing device identified via the one or more machine-learned models, and obtain the display screen information associated with the display screen of the biometric sensing computing device based on the device information

Aspects of the above-described example embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy disks, and magnetic tape; optical media such as CD ROM disks, Blue-Ray disks, and DVDs; magneto-optical media such as optical discs; and other hardware devices that are specially configured to store and perform program instructions, such as semiconductor memory, read-only memory (ROM), random access memory (RAM), flash memory, USB memory, and the like. Examples of program instructions include both machine code, such as produced by a compiler, and files containing higher level code that may be executed by the computer using an interpreter. The program instructions may be executed by one or more processors. The described hardware devices may be configured to act as one or more software modules in order to perform the operations of the above-described embodiments, or vice versa. In addition, a non-transitory computer-readable storage medium may be distributed among computer systems connected through a network and computer-readable codes or program instructions may be stored and executed in a decentralized manner. In addition, the non-transitory computer-readable storage media may also be embodied in at least one application specific integrated circuit (ASIC) or Field Programmable Gate Array (FPGA).

Each block of the flowchart illustrations may represent a unit, module, segment, or portion of code, which comprises one or more executable instructions for implementing the specified logical function(s). It should also be noted that in some alternative implementations, the functions noted in the blocks may occur out of order. For example, two blocks shown in succession may in fact be executed substantially concurrently (simultaneously) or the blocks may sometimes be executed in the reverse order, depending upon the functionality involved.

While the disclosure has been described with respect to various example embodiments, each example is provided by way of explanation, not limitation of the disclosure. Those skilled in the art, upon attaining an understanding of the foregoing, can readily produce alterations to, variations of, and equivalents to such embodiments. Accordingly, the disclosure does not preclude inclusion of such modifications, variations and/or additions to the disclosed subject matter as would be readily apparent to one of ordinary skill in the art. For example, features illustrated or described as part of one embodiment can be used with another embodiment to yield a still further embodiment. Thus, it is intended that the disclosure covers such alterations, variations, and equivalents.

1. A computing device, comprising:
one or more memories configured to store one or more instructions; and
one or more processors configured to execute the one or more instructions stored in the one or more memories to:
obtain display screen information associated with a display screen of a biometric sensing computing device,
obtain an image including the display screen displaying biometric information associated with a user of the biometric sensing computing device,
execute a digital display function based on the display screen information to determine biometric content information from the image corresponding to the biometric information, and
store the biometric content information determined by executing the digital display function.

2. The computing device of claim 1, the one or more processors being configured to execute the one or more instructions stored in the one or more memories to:
obtain device information associated with the biometric sensing computing device using an image of the biometric sensing computing device, and
obtain the display screen information associated with the display screen of the biometric sensing computing device based on the device information.

3. The computing device of claim 1, the one or more processors being configured to execute the one or more instructions stored in the one or more memories to:
receive information associated with a user input providing device information associated with the biometric sensing computing device, and
obtain the display screen information associated with the display screen of the biometric sensing computing device based on the device information.

4. The computing device of claim 2, the device information including at least one of a device part number, a device model number, a device name, or a device manufacturer.

5. The computing device of claim 1, the display screen information including at least one of a resolution of the display screen, a size of the display screen, color information of the display screen, font information of the display screen, a type of the display screen, layout information of the display screen, tolerance information associated with the display screen, or text placement information of the display screen.

6. The computing device of claim 1, the one or more processors being configured to execute the one or more instructions stored in the one or more memories to perform perspective correction with respect to the image displayed on the display screen before executing the digital display function.

7. The computing device of claim 1, the one or more processors being configured to execute the one or more instructions stored in the one or more memories to select one or more digital display functions to determine the biometric content information from the image based on biometric device capabilities associated with the biometric sensing computing device.

8. The computing device of claim 7, the biometric device capabilities associated with the biometric sensing computing device including at least one capability to: generate an electrocardiogram, generate a photoplethysmogram, measure heart rate, measure blood pressure, measure oxygen saturation, measure respiration rate, measure body temperature, measure physical activity, measure a sleep metric, or measure electrical conductance.

9. The computing device of claim 1, the one or more processors being configured to execute the one or more instructions stored in the one or more memories to determine the digital display function to be executed based on whether a display output space provided on the display screen includes a discrete display output space and/or a continuous display output space.

10. The computing device of claim 9, the one or more processors being configured to execute the one or more instructions stored in the one or more memories to, when a first portion of the display output space provided on the display screen includes the discrete display output space, execute a first digital display function with respect to the first portion to:
compare the image including the biometric information with each of a plurality of possible discrete values which can be output with respect to the first portion of the display output space, to thereby determine a similarity metric value for each comparison,
select a discrete value among the plurality of possible discrete values that is associated with a highest degree of similarity based on the similarity metric values determined from the comparisons, and
store the discrete value associated with the highest degree of similarity as the biometric content information corresponding to the biometric information of the user.

11. The computing device of claim 10, the one or more processors being configured to execute the one or more instructions stored in the one or more memories to, when a second portion of the display output space provided on the display screen includes the continuous display output space, execute a second digital display function with respect to the second portion to:
determine a type of biometric signal displayed in the image,
apply a computer-implemented spline fitting method to reconstruct the biometric signal displayed in the image to obtain a reconstructed biometric signal, based on the type of biometric signal displayed in the image, and
store the reconstructed biometric signal as the biometric content information corresponding to the biometric information of the user.

12. The computing device of claim 11, the one or more processors being configured to execute the one or more instructions stored in the one or more memories to, when the reconstructed biometric signal includes one or more missing data portions, apply a computer-implemented spline interpolation method to fully reconstruct the biometric signal, the computer-implemented spline interpolation method being based on the type of biometric signal displayed in the image.

13. The computing device of claim 10, the one or more processors being configured to execute the one or more instructions stored in the one or more memories to, when a first portion of the display output space provided on the display screen includes the discrete display output space, execute a first digital display function to:
compare the image including the biometric information with each of a plurality of possible discrete values which can be output with respect to the first portion of the display output space, to thereby determine a similarity metric value for each comparison,
identify a discrete value among the plurality of possible discrete values that is associated with a highest degree of similarity based on the similarity metric values determined from the comparisons,
when a confidence level associated with the discrete value associated with the highest degree of similarity is less than a threshold confidence level, provide an output requesting feedback from the user confirming a configuration of the display output space, and
when the confidence level associated with the discrete value associated with the highest degree of similarity is more than the threshold confidence level, store the discrete value associated with the highest degree of similarity as the biometric content information corresponding to the biometric information of the user.

14. The computing device of claim 13, the output requesting the feedback from the user confirming the configuration of the display output space including enabling the user to provide an input to the computing device indicating an actual layout of the display output space as presented on the biometric sensing computing device.

15. The computing device of claim 1, the one or more processors being configured to execute the one or more instructions stored in the one or more memories to automatically store the biometric content information in a remote server computing system in response to the computing device connecting to a network.

16. (canceled)

17. A computer-implemented method, comprising:
obtaining display screen information associated with a display screen of a biometric sensing computing device;
obtaining an image including the display screen displaying biometric information associated with a user of the biometric sensing computing device;
executing a digital display function based on the display screen information to determine biometric content information from the image corresponding to the biometric information; and
storing the biometric content information determined by executing the digital display function.

18. The computer-implemented method of claim 17, further comprising:
obtaining device information associated with the biometric sensing computing device using an image of the biometric sensing computing device; and
obtaining the display screen information associated with the display screen of the biometric sensing computing device based on the device information.

19. The computer-implemented method of claim 17, further comprising determining the digital display function to be executed based on whether a display output space provided on the display screen includes a discrete display output space and/or a continuous display output space.

20. (canceled)

21. A computing device, comprising:
one or more cameras;
one or more memories configured to store one or more instructions; and
one or more processors configured to execute the one or more instructions stored in the one or more memories to:
obtain display screen information associated with a display screen of a biometric sensing computing device,
capture, using the one or more cameras, an image of the display screen of the biometric sensing computing device, the display screen displaying biometric information associated with a user,
process the image of the display screen by implementing one or more machine-learned models to determine biometric content information from the image corresponding to the biometric information, and
store the biometric content information determined by processing the image.

22. The computing device of claim 21, the one or more processors being configured to execute the one or more instructions stored in the one or more memories to:
implement one or more machine-learned models to identify the biometric sensing computing device, using an image of the biometric sensing computing device captured by the one or more cameras,
obtain device information associated with the biometric sensing computing device using based on the biometric sensing computing device identified via the one or more machine-learned models, and
obtain the display screen information associated with the display screen of the biometric sensing computing device based on the device information.
